(19) United States
(12) Patent Application Publication
(10) Pub. No.: US 2023/0023667 A1
(43) Pub. Date: Jan. 26, 2023

(54) SYSTEM FOR ANALYZING EFFECTS OF MINERAL CRYSTALS TO HUMAN BODY AND METHOD FOR PERFORMING THE SAME

(71) Applicants: Ching Lee, Taipei City (TW); Ruey Yuan Lee, Taipei City (TW)

(72) Inventors: Ching Lee, Taipei City (TW); Ruey Yuan Lee, Taipei City (TW)

(21) Appl. No.: 17/380,052

(22) Filed: Jul. 20, 2021

(51) Int. Cl. A61B 5/377; A61B 5/00; A61B 5/374

(52) U.S. Cl. CPC A61B 5/377; A61B 5/6803; A61B 5/374; A61N 2/006

(57) ABSTRACT

A system for analyzing effects of mineral crystals to a human body and the method for performing the system comprises a helmet, a brainwave; a brainwave transceiver; a processor unit. The processor unit includes a processor transceiver, a brainwave calculating unit, a testing person database, a physiological and psychological effect database, a brainwave comparison module. In test, a specific mineral crystal is located in different distance from a testing person to test effects of the mineral crystal to the testing person; the testing person wears the helmet and the brainwave transceiver transmits measured brainwaves to the processor unit; and then the processor unit calculates statistical data about the brainwaves, and from the physiological and psychological effect database, effects of the mineral crystal to the testing person can be determined.

### FIELD OF THE INVENTION

The present invention is related to effects of mineral crystals to human body, and in particular to a system for analyzing effects of mineral crystals to a human body and a method for performing the same.

### BACKGROUND OF THE INVENTION

Mineral crystals are classified as natural mineral crystals and artificial mineral crystals. Each kind of mineral crystals has its own distribution of electric waves and magnetic waves. Generally, most parts of natural mineral crystals have minor effects to human body, while many kinds of artificial mineral crystals have deep effects to human bodies. A human body is conductive and can emit brainwaves. If the electromagnetic waves from a mineral crystal cover the range of brainwaves, or if the strength of the electromagnetic wave from the mineral crystal is strong, the physiological and psychological effects will generate for the person near the mineral crystal.

The Delta waves, Theta waves, High/Low Alpha waves, High/Low Beta waves and High/Low Gamma waves of right brain and left brain have different physiological and psychological effects to human bodies. Therefore, by measuring these brainwaves and calculating different numerical operations, the physiological and psychological effects of the testing person can be obtained. However, all these results have been researched for a long time and can be got easily from many papers.

With the progressions of technologies, more and more natural mineral crystals and artificial mineral crystals are developed and are located near the human bodies, as a result, these mineral crystals are possibly to affect the human bodies. Therefore, there is a need for a novel way which can get the physiological and psychological effects of these mineral crystals to the human bodies easily and quickly.

### SUMMARY OF THE INVENTION

Accordingly, the object of the present invention is to provide a system for analyzing effects of mineral crystals to a human body and the method based on the system, wherein the effects of mineral crystals are tested, such as the effect of meditation or attention, etc. Therefore, by the system of the present invention, the bad or good effect of the mineral crystal can be obtained quickly and precisely.

To achieve above object, the present invention provides a system for analyzing effects of mineral crystals to a human body, comprising:

a helmet for detecting brainwave signals; in use, the helmet being installed on a head of a testing person; the helmet comprising:

a ring installed on the head of a testing person;

a brainwave detector positioned on the ring;

a brainwave transceiver connected to the brainwave detector for transferring the brainwave signals detected by the brainwave detector;

a processor unit for processing effects to brainwaves of a testing person under an environment with mineral crystals or without mineral crystal, in that, by comparing brainwave signals, the physiological or psychological effect to the testing person can be found;

the processor unit including;

a processor transceiver signally connected to the brainwave transceiver of the brainwave detector for receiving signals from the brainwave transceiver;

a brainwave calculating unit connected to the processor transceiver for calculating statistical values;

a testing person database for storing brainwave data and related statistical values of the brainwave data of testing persons without the effect of the mineral crystals;

a physiological and psychological effect database of the brainwave change storing the physiological or psychological effect values of the brainwave change, these effect values being called as historical data which are obtained from many prior research papers; if the mean value of the distribution after affected by a mineral crystal;

a brainwave comparison module is connected to the processor transceiver, the testing person database and the physiological and psychological affect database; in one test, the brainwave comparison module comparing brainwave data from the brainwave calculating unit with the statistical data stored in the testing person database for assuring the difference therebetween; in that, the brainwave data means the data of different kinds of brainwaves and the related statistical data; the test means that a specific mineral crystal is placed near the testing person with a specific distance therefrom; the distance causes that the mineral crystal can interfere the brainwaves of the testing person;

the brainwave comparison module also searching a respective data from the physiological and psychological effect database based on a brainwave data as a mineral crystal is placed near the testing person, which is obtained from the brainwave calculating unit; and wherein in test, a specific mineral crystal is located in different distance from a testing person to test effects of the mineral crystal to the testing person; the testing person wears the helmet and the brainwave transceiver transmits measured brainwaves to the processor unit; and then the processor unit calculates statistical data about the brainwaves, and from the physiological and psychological effect database, effects of the mineral crystal to the testing person can be determined.

Furthermore, the present invention provides a method for analyzing effects of mineral crystals to a human body, comprising the steps of:

Step 101: a testing person wearing a helmet which outputs a series of brainwaves which includes the Delta waves, Theta waves, High/Low Alpha waves, High/Low Beta waves and High/Low Gamma waves of right brain and left brain; these brainwaves being received by a brainwave transceiver and being transferred to the processor unit; then the brainwave calculating unit in the processor unit calculating the statistical data for each kind of brainwaves; the values of these brainwaves and statistical data being stored in the testing person database, which are used as records under a condition that the testing person is not interfered by any mineral crystal;.

Step 102: placing a mineral crystal to be near the testing person with a predetermined distance, in that the mineral crystal generates interference to the testing person; since the mineral crystal generates electric and magnetic fields which interfere the brainwaves of the testing person; then the testing person wearing the helmet and the helmet outputs a series of brainwaves which are transferred to the processor unit through the brainwave transceiver; then the brainwave comparison module calculating the statistical data for each brainwave;

Step 103: using a brainwave comparison module to compare the variations from step 102 with the variations from step 101 stored in the testing person database 40 for different kinds of brainwaves;

Step 104: if in step 103, the variations derived from step 102 being smaller than those derived from 101; then the means from step 102 being used to find a history data stored in the physiological and psychological affect database so as to know how the mineral crystal affects the testing person;

Step 105: if in step 103, the variations derived from step being greater than those obtained from step 101, the step 102 is repeated several times under the interferences of mineral crystals; and then the means from these several repeated times are averaged to get an averaged mean; the brainwave comparison module using the averaged mean to find a history data stored in the physiological and psychological affect database so as to know how the mineral crystal affect the testing person.

### BRIEF DESCRIPTION OF THE DRAWINGS

FIG. 1 is a structural block diagram of the present invention.

FIG. 2 is a schematic view showing the helmet of the present invention.

FIG. 3 is a schematic view showing that a mineral crystal is located near a testing person.

FIG. 4 is a step diagram from performing the steps of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

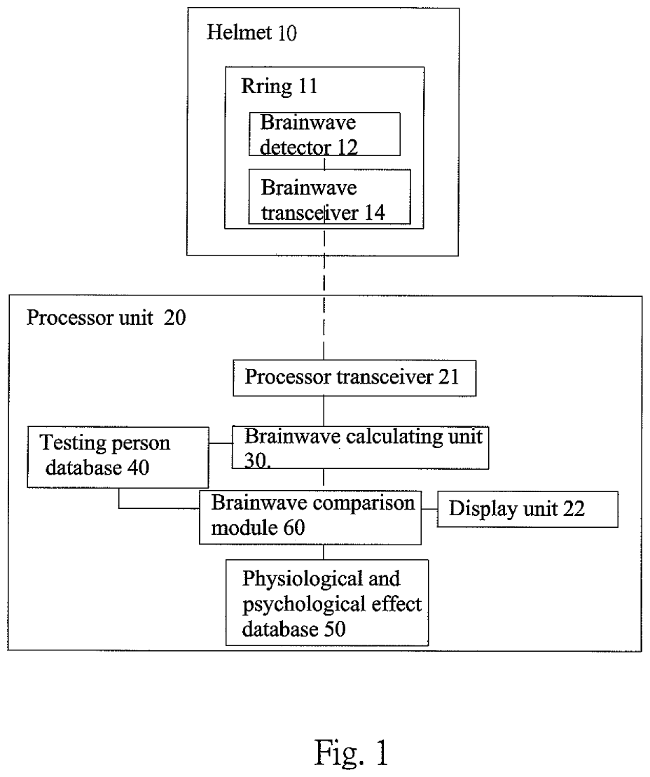

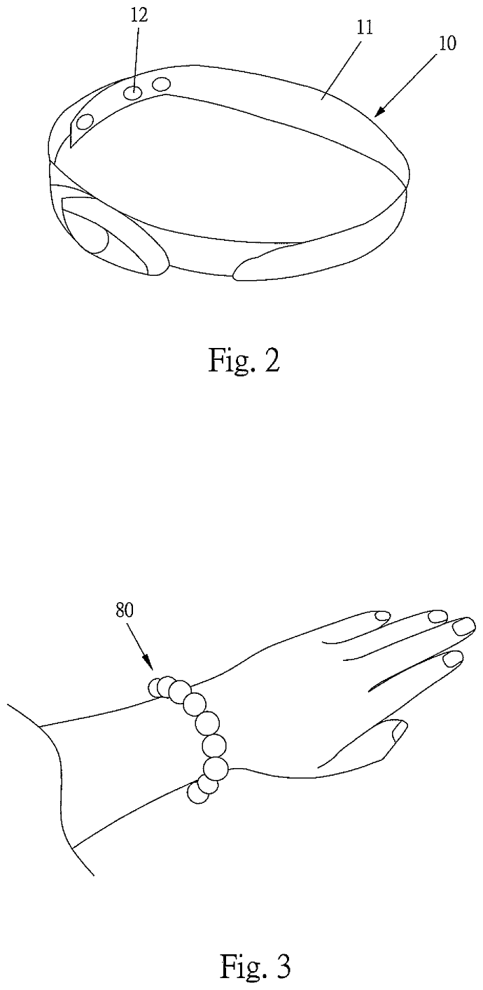

With reference to FIGS. 1 to 3, the system of the present invention is illustrated. The present invention includes the following elements. A helmet 10 (referring to FIG. 2) serves for detecting brainwave signals.

In use, the helmet 10 is installed on a head of a testing person.

With reference to FIG. 1, the helmet 10 includes the following elements.

A ring 11 is installed on the head of the testing person.

A brainwave detector 12 is positioned on the ring 11 near the head of the testing person.

A brainwave transceiver 14 is connected to the brainwave detector 12 for transferring the brainwave signals detected by the brainwave detector 12. The brainwave transceiver 14 is for example a Bluetooth transceiver.

A processor unit 20 for processing effects of mineral crystal to brainwaves of the testing person is used to make AI (artificial intelligence) processing about brainwave signals from the testing person which is under an environment with mineral crystals or without mineral crystal, in that, by comparing brainwave signals, the physiological or psychological effect to the testing person can be found.

The processor unit 20 includes following elements.

A processor transceiver 21 is signally connected to the brainwave transceiver 14 of the brainwave detector 12 for receiving signals from the brainwave transceiver 14.

A brainwave calculating unit 30 is connected to the processor transceiver 21 for calculating statistical values based specific algorithm. The statistical values are such as mean values, variations, maximums, etc. These are calculated based on the Delta waves, Theta waves, High/Low Alpha waves, High/Low Beta waves and High/Low Gamma waves of right brain and left brain.

A testing person database 40 serves for storing brainwave data and related statistical values of the brainwave data of testing persons without the effect of the mineral crystals. The statistical values are such as means, variations, maximums, etc.

A physiological and psychological effect database 50 of the brainwave change stores the physiological or psychological effect values of the brainwave change, these effect values are called historical data which are obtained from many prior research papers. The brainwave includes Delta waves, Theta waves, High/Low Alpha waves, High/Low Beta waves and High/Low Gamma waves of right brain and left brain. The effects are such as brainwave meditation, attention, etc. For example, a Theta brainwave is generally has a statistical distribution. If the mean value of the distribution after affected by a mineral crystal (a mineral crystal is placed near a testing person) is shifted with a value of 5 unit from another means value of a distribution without effect of the mineral crystal, it means that the attention of the testing person is not concentrated. This affect can be got from many research papers.

A brainwave comparison module 60 is connected to the processor transceiver 21, the testing person database 40 and the physiological and psychological affect database 50. In one test, the brainwave comparison module 60 compares brainwave data from the brainwave calculating unit 30 with the statistical data stored in the testing person database 40 for assuring the difference therebetween on the EEG(electroencephalogram, waveform of brainwave). In that, the brainwave data means the data of different kinds of brainwaves and the related statistical data. The statistical data is for example means, variations, maximums, etc. The test means that a specific mineral crystal 80 is placed near the testing person with a specific distance therefrom. The distance means a distance which causes that the mineral crystal 80 can interfere the brainwaves of the testing person. The distance may be 0, that is to say, the mineral crystal 80 is directly in contact with the testing person, such as wearing on the hand, see FIG. 3, or the mineral crystal 80 is placed with a distance of 5 to 25 centimeters to the testing person.

The brainwave comparison module 60 also searches a respective data from the physiological and psychological effect database 50 based on a brainwave data as a mineral crystal 80 is placed near the testing person, which is obtained from the brainwave calculating unit 30.

A display unit 22 is connected to the processor unit for displaying the comparing and testing results.

The processor unit 20 may be installed in various kinds of electronic devices, such as a computer, a mobile phone, a tablet computer, etc.

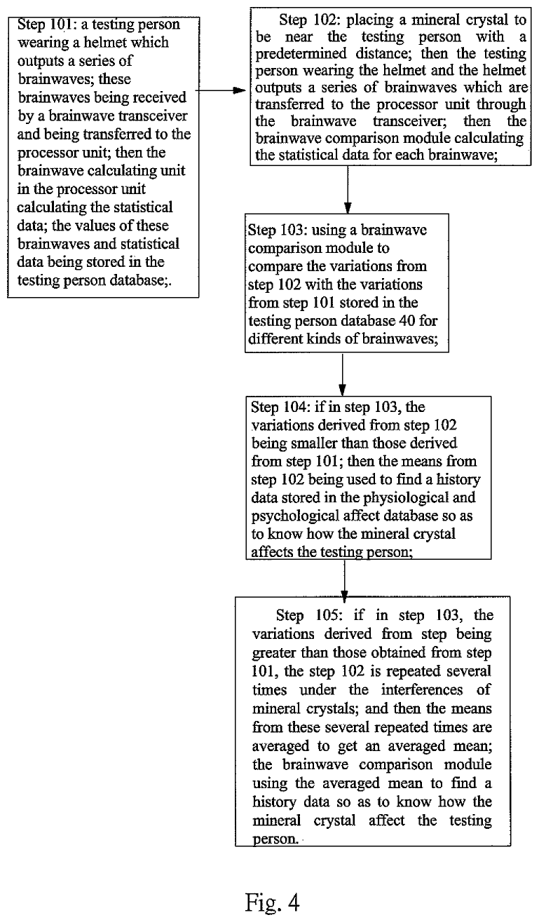

With reference to FIG. 4, in the present invention, a process for testing and comparing with the above mentioned brainwave analysis system is described hereinafter with the steps of:.

Step 101: a testing person wearing a helmet 10 which outputs a series of brainwaves which includes the Delta waves, Theta waves, High/Low Alpha waves, High/Low Beta waves and High/Low Gamma waves of right brain and left brain. These brainwaves are received by the brainwave transceiver 14 and are transferred to the processor unit 20. Then the brainwave calculating unit 30 in the processor unit 20 calculates the statistical data for each kind of brainwaves, such as means, variations (or standard deviations), maximums, etc. The values of these brainwaves and statistical data are stored in the testing person database 40, which are used as records under a condition that the testing person is not interfered by any mineral crystal 80.

Step 102: Placing a mineral crystal 80 to be near the testing person with a predetermined distance, in that the mineral crystal 80 generates interference to the testing person. Since the mineral crystal 80 generates electric and magnetic fields which interfere the brainwaves of the testing person. Then the testing person wearing the helmet 10 and the helmet 10 will output a series of brainwaves which are transferred to the processor unit 20 through the brainwave transceiver 14; then the brainwave comparison module 60 calculating the statistical data for each brainwave.

In above step 102, the distance between the testing person and the mineral crystal can be zero, or be 5 to 25 centimeters.

Step 103: Using the brainwave comparison module 60 to compare the variations from step 102 with the variations from step 101 stored in the testing person database 40 for different kinds of brainwaves.

Step 104: if in step 103, the variations derived from step 102 (with interference of mineral crystal 80) is smaller than those derived from step 101 (without interference of mineral crystal). Then the means from step 102 is used to find a history data stored in the physiological and psychological affect database 50 so as to know how the mineral crystal 80 affect the testing person (may be only a part of physiological or psychological affect).

Step 105: if in step 103, the variations derived from step 102 are greater than those obtained from step 101, that is, the distributions of the brainwaves are greater under interference of mineral crystal 80 and the physical meanings of the means are low preciseness. Therefore, the step 102 is repeated several times under the interferences of mineral crystals, for example, four times. The means from the several calculations are averaged to get an averaged mean. The brainwave comparison module 60 uses the averaged mean to find a history data stored in the physiological and psychological affect database 50 so as to know how the mineral crystal 80 affect the testing person (may be only a part of physiological or psychological affect).

Advantages of the present invention are that the effects of mineral crystals are tested, such as the effect of meditation or attention, etc. Therefore, by the system of the present invention, the bad or good effect of the mineral crystal can be obtained quickly and precisely.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A system for analyzing effects of mineral crystals to a human body, comprising:
a helmet for detecting brainwave signals; in use, the helmet being installed on a head of a testing person; the helmet comprising:
a ring installed on the head of a testing person;
a brainwave detector positioned on the ring;
a brainwave transceiver connected to the brainwave detector for transferring the brainwave signals detected by the brainwave detector;
a processor unit for processing effects to brainwaves of a testing person under an environment with mineral crystals or without mineral crystal, in that, by comparing brainwave signals, the physiological or psychological effect to the testing person can be found;
the processor unit including;
a processor transceiver signally connected to the brainwave transceiver of the brainwave detector for receiving signals from the brainwave transceiver;
a brainwave calculating unit connected to the processor transceiver for calculating statistical values;
a testing person database for storing brainwave data and related statistical values of the brainwave data of testing persons without the effect of the mineral crystals;
a physiological and psychological effect database of the brainwave change storing the physiological or psychological effect values of the brainwave change, these effect values being called as historical data which are obtained from many prior research papers; if the mean value of the distribution after affected by a mineral crystal;
a brainwave comparison module is connected to the processor transceiver, the testing person database and the physiological and psychological affect database; in one test, the brainwave comparison module comparing brainwave data from the brainwave calculating unit with the statistical data stored in the testing person database for assuring the difference therebetween; in that, the brainwave data means the data of different kinds of brainwaves and the related statistical data; the test means that a specific mineral crystal is placed near the testing person with a specific distance therefrom; the distance causes that the mineral crystal can interfere the brainwaves of the testing person;
the brainwave comparison module also searching a respective data from the physiological and psychological effect database based on a brainwave data as a mineral crystal is placed near the testing person, which is obtained from the brainwave calculating unit; and
wherein in test, a specific mineral crystal is located in different distance from a testing person to test effects of the mineral crystal to the testing person; the testing person wears the helmet and the brainwave transceiver transmits measured brainwaves to the processor unit; and then the processor unit calculates statistical data about the brainwaves, and from the physiological and psychological effect database, effects of the mineral crystal to the testing person can be determined.

2. The system for analyzing effects of mineral crystals to a human body as claimed in claim 1, wherein the statistical data includes at least one of means, variations, maximum.

3. The system for analyzing effects of mineral crystals to a human body as claimed in claim 1, wherein the brainwaves outputted from the helmet includes the Delta waves, Theta waves, High/Low Alpha waves, High/Low Beta waves and High/Low Gamma waves of right brain and left brain.

4. The system for analyzing effects of mineral crystals to a human body as claimed in claim 1, wherein the physiological and psychological effect database stores data about meditation and attention.

5. The system for analyzing effects of mineral crystals to a human body as claimed in claim 1, wherein the distance between the mineral crystal and the testing person is zero, that is, the mineral crystal is in contact with the testing person.

6. The system for analyzing effects of mineral crystals to a human body as claimed in claim 1, wherein the distance between the mineral crystal and the testing person is 5 centimeter to 25 centimeter.

7. The system for analyzing effects of mineral crystals to a human body as claimed in claim 1, further comprising:
a display unit connected to the processor unit for displaying the comparing and testing results.

8. A method for analyzing effects of mineral crystals to a human body, comprising the steps of:
Step 101: a testing person wearing a helmet which outputs a series of brainwaves which includes the Delta waves, Theta waves, High/Low Alpha waves, High/Low Beta waves and High/Low Gamma waves of right brain and left brain; these brainwaves being received by a brainwave transceiver and being transferred to the processor unit; then the brainwave calculating unit in the processor unit calculating the statistical data for each kind of brainwaves; the values of these brainwaves and statistical data being stored in the testing person database, which are used as records under a condition that the testing person is not interfered by any mineral crystal;.
Step 102: placing a mineral crystal to be near the testing person with a predetermined distance, in that the mineral crystal generates interference to the testing person; since the mineral crystal generates electric and magnetic fields which interfere the brainwaves of the testing person; then the testing person wearing the helmet and the helmet outputs a series of brainwaves which are transferred to the processor unit through the brainwave transceiver; then the brainwave comparison module calculating the statistical data for each brainwave;
Step 103: using a brainwave comparison module to compare the variations from step 102 with the variations from step 101 stored in the testing person database 40 for different kinds of brainwaves;
Step 104: if in step 103, the variations derived from step 102 being smaller than those derived from 101; then the means from step 102 being used to find a history data stored in the physiological and psychological affect database so as to know how the mineral crystal affects the testing person;
Step 105: if in step 103, the variations derived from step being greater than those obtained from step 101, the step 102 is repeated several times under the interferences of mineral crystals; and then the means from these several repeated times are averaged to get an averaged mean; the brainwave comparison module using the averaged mean to find a history data stored in the physiological and psychological affect database so as to know how the mineral crystal affect the testing person.

9. The method as claimed in claim 8, wherein the distance between the mineral crystal and the testing person is zero, that is, the mineral crystal is in contact with the testing person

10. The method as claimed in claim 8, wherein the distance between the mineral crystal and the testing person is 5 centimeters to 25 centimeters.

11. The method as claimed in claim 8, wherein the brainwaves outputted from the helmet includes the Delta waves, Theta waves, High/Low Alpha waves, High/Low Beta waves and High/Low Gamma waves of right brain and left brain.

12. The method as claimed in claim 8, wherein the statistical data includes at least one of means, variations and maximums.
